(19) United States
(12) Patent Application Publication
(10) Pub. No.: US 2026/0090924 A1
(43) Pub. Date: Apr. 2, 2026

(54) CELLULOSIC TAMPON APPLICATOR

(71) Applicant: THE PROCTER & GAMBLE COMPANY, Cincinnati, OH (US)

(72) Inventors: Khalid QURESHI, Mason, OH (US); Evan Joseph DURLING, West Chester, OH (US); Ryo MINOGUCHI, Cincinnati, OH (US)

(21) Appl. No.: 19/345,552

(22) Filed: Sep. 30, 2025

Related U.S. Application Data
(60) Provisional application No. 63/701,853, filed on Oct. 1, 2024.

(51) Int. Cl. A61F 13/26; A61F 13/20; A61F 13/34

(52) U.S. Cl. CPC A61F 13/266; A61F 13/2097; A61F 13/34

(57) ABSTRACT

A tampon product is presented that includes an applicator including an outer member and an inner member slidably engaged with the outer member. The outer member includes an outer surface surrounding a longitudinal applicator axis, a barrel region, and a grip region. The grip region comprises greater than four circumferentially formed faces. One or more layers defining the applicator include cellulosic material.

### CROSS REFERENCE TO RELATED APPLICATIONS

This application claims the benefit, under 35 U.S.C. § 119 (e), to U.S. Provisional Application No. 63/701,853, filed Oct. 1, 2024, the entire disclosure of which is fully incorporated by reference herein.

### FIELD

The present disclosure relates to tampon applicators, and more particularly, to tampon applicators made of cellulosic material.

### BACKGROUND

Consumer products or portions thereof that have components derived from petroleum (such as but not limited to plastics), are currently becoming subject to increased regulation by governments in a number of countries, and growing disfavor in some segments of the consumer market. Some reasons for this relate generally to the consumer perception of certain materials having a relatively adverse impact on the environment.

Tampons have been used for many years to manage menstrual discharge. Many consumers prefer tampons provided inside disposable applicators (rather than tampons configured to be inserted via a finger (“digitally”) without use of an applicator). The typical tampon applicator is configured to house and protect the new tampon from deformation and/or contamination prior to use, and then to facilitate application of the tampon when its use is desired.

Currently, many tampon applicators or parts thereof are formed of thermoplastics such as polyethylene. The properties of thermoplastics and known associated forming/molding technologies enable a manufacturer to efficiently make an applicator having relatively sophisticated features, which may be provided for purposes of functionality, convenience, comfort during use, ergonomics and/or aesthetic appeal, thereby making it attractive and convenient to consumers. FIG. 1A depicts a conventional plastic applicator 10 with a barrel region 12 having an external diameter 13, a plunger 30 having an external diameter 31 and a grip region 18 positioned between the barrel region 12 and plunger 30. As shown in FIG. 1A, the external diameter 31 of the plunger 30 is noticeably less than the external diameter 13 of the barrel region 12.

Other types of tampon applicators currently marketed are formed of paperboard or cardboard. While such applicators reduce reliance on and use of materials derived from petroleum as compared to plastic applicators, currently, these cardboard applicators do not provide the same functionality, comfort during use, ergonomics and/or aesthetic appeal as plastic applicators. FIG. 1B depicts a conventional cardboard applicator 10′ including a barrel region 12′ having an external diameter 13′, a plunger 30′ having an external diameter 31′ and a grip region 18′ between the barrel region 12′ and the plunger 30′. Unlike the plastic applicator 10 of FIG. 1A, the external diameter 31′ of the plunger 30′ is relatively close the external diameter 13′ of the barrel region 12′. FIG. 1C depicts another conventional cardboard applicator 10′ where the grip region 18′ includes four impressions which are spaced apart by the outer surface 42′ of the grip region 18′.

Accordingly, there remain opportunities for improvements in cardboard applicators design and functionality such that carboard applicators more closely emulate their plastic applicator counterparts.

### SUMMARY

The inventors of the present invention recognized various advantages of the conventional plastic applicator that are absent in conventional cardboard applicators. These include features of the plastic applicators which facilitate functionality, convenience, comfort during use, ergonomics and/or aesthetic appeal. The inventors also recognized that recent environmental regulations in various geographical territories that promote the use of cardboard material over plastic material will likely cause a shift in consumer demand towards cardboard applicators. Thus, the inventors developed an improved cardboard applicator herein, which achieves many of the advantages of plastic applicators that are absent in conventional cardboard applicators. In order to achieve this improved cardboard applicator, the inventors necessarily overcame various design obstacles not encountered in manufacturing plastic applicators.

In some embodiments, a tampon product is presented that includes an applicator including an outer member and an inner member slidably engaged with the outer member. The outer member includes an outer surface surrounding a longitudinal applicator axis, a barrel region, and a grip region. The grip region comprises greater than four circumferentially formed faces.

In some embodiments, a tampon product is presented that comprises an applicator that includes an outer member and an inner member slidably engaged with the outer member. The outer member includes an outer surface, a barrel region, and a grip region. The applicator includes one or more layers, where at least one of the layers includes cellulosic material. The grip region comprises greater than four faces.

In some embodiments, a tampon product is presented that is similar to the second set of embodiments except that each of the one or more layers includes cellulosic material.

### BRIEF DESCRIPTION OF THE DRAWINGS

FIG. 1A is a front view of a conventional plastic applicator including a barrel region and a plunger.

FIG. 1B is a front view of a conventional cardboard applicator including a barrel region and a plunger.

FIG. 1C is a front view of a conventional cardboard applicator.

FIG. 2A is a perspective view of a tampon product with an applicator including a barrel region and a plunger.

FIG. 2B is a longitudinal side view of the tampon product of FIG. 2A taken along the line 2B-2B.

FIG. 2C is a longitudinal side view of a plunger of the tampon product of FIG. 2A with a flange at a second end region thereof.

FIG. 2D is a perspective view of a plunger of the tampon product with a flange at a first end region and second end region thereof.

FIG. 3A is a perspective view of a machine used to form circumferential faces in the grip region of an applicator.

FIG. 3B is a perspective view of a plurality of forming members of the machine of FIG. 3A that move radially inward to form the circumferential faces in the grip region of the applicator.

FIGS. 4A through 4C are perspective and top views of a plurality of circumferential faces and ribs therebetween formed in the grip region of an applicator.

FIG. 4D is a cross-sectional view of the grip region of FIG. 4A taken along the line 4D-4D.

FIG. 4E is a cross-sectional view of a rib of the cross-sectional view of the grip region of FIG. 4D.

FIG. 4F is a cross-sectional view of a rib of the cross-sectional view of the grip region.

FIG. 4G is a cross-sectional view of the grip region of FIG. 4C taken along the line 4G-4G.

FIG. 4H is a cross-sectional view of the grip region of FIG. 4A taken along the line 4H-4H.

FIGS. 5A through 5C are perspective views of an end region of the barrel region of an applicator.

FIGS. 6A through 6C are side views of an applicator including a barrel region and a plunger with circumferential faces formed in both the barrel region and the plunger.

FIG. 7A is a cross-sectional side view of a plurality of material layers that define the applicator.

FIG. 7B is a side view of an applicator including a spirally wound barrel region.

FIG. 7C is a side view of an applicator including a convolutely wound barrel region.

### DETAILED DESCRIPTION

The following term explanations may be useful in understanding the present disclosure.

As used herein the term “tampon” refers to any type of absorbent structure which is configured to be inserted into the vaginal cavity for the interception and absorption of fluid therefrom. Typically, a tampon includes a pledget structure including a quantity of absorbent material, often absorbent fibrous material, which pledget structure has been bunched, folded and/or compressed in one or more lateral/radial directions, the longitudinal direction, or both, via application of pressure, heat and/or moisture control, in order to provide a formed tampon having a size, shape (typically approximately cylindrical) and stability of form to facilitate insertion into the vagina. A tampon which has been so formed is referred to herein has a “self-sustaining” form. The degree of compression, heat and moisture control applied to the pledget is sufficient such that in the subsequent absence of the external forces and absence of substantial contact with moisture, the pledget will tend to retain its general formed shape and size. It will be understood by persons of ordinary skill in the art that this self-sustaining form typically does not persist following insertion of the tampon. Once the tampon is inserted and begins to contact and absorb fluid, the pledget will swell with absorbed fluid and lose its self-sustaining form.

As used herein the terms “pledget” or “tampon pledget” are intended to be interchangeable and refer to a structure including absorbent material configured to perform the primary function of the tampon, absorption of menstrual fluid. A tampon pledget is sometimes referred to as a tampon blank, or a softwind, and the term “pledget” is intended to include structures designated by such terms as well.

As used herein “vaginal cavity” refers to the internal space within the genitalia of the human female, located between the introitus of the vagina (sometimes referred to as the sphincter of the vagina) and the cervix.

With respect to a tampon and an applicator, the “longitudinal” direction is the ordinary general direction of ejection from an applicator; and also corresponds with the ordinary general direction of insertion of a tampon and applicator into, and their withdrawal from, the vaginal cavity in normal use. For a completely manufactured, pre-use tampon that has a pledget with a generally cylindrical or capsule-shaped self-sustaining form, the longitudinal axis of the form lies generally or approximately along the longitudinal direction. A “radial” or “circumferential” direction is a direction perpendicular to the longitudinal direction. The “circumferential” direction is perpendicular to the longitudinal direction and the radial direction, and perpendicular to the z direction (defined below). Unless otherwise specified, references to “length” herein refer to a dimension along the longitudinal direction; references to “width” herein refer to a dimension along the circumferential direction.

With respect to a tampon and an applicator, the term “forward” refers to a longitudinal direction of movement during normal insertion of the tampon and/or applicator by a user, and refers to portions of the tampon or applicator that lie closer to and/or enter the vaginal cavity earlier than other portions during normal insertion. Conversely, the term “rearward” refers to a longitudinal direction of movement during normal withdrawal of the tampon and/or applicator by a user, and refers to portions of the tampon or applicator that lie farther from and/or exit the vaginal cavity earlier than other portions during normal withdrawal.

A “nonwoven,” “nonwoven web,” “nonwoven web material,” or “nonwoven fabric” is a cloth-like web material (or portion or section thereof) formed predominantly of fibers that are neither knitted nor woven, but rather, laid down and accumulated to a desired basis weight, then consolidated and held together to form a web, via one or any combination of calendering, thermal and/or compression bonding, bonding via use of a binder, heating (via, e.g., heated air driven through an accumulation of fibers) or hydroentangling (spunlace). The predominant fibers may be natural fibers harvested from plant material (e.g., cotton) (but excluding tree wood pulp), semi-synthetic (e.g., rayon, lyocell, viscose), or synthetic (e.g., fibers spun from molten thermoplastic polymer resin(s)), or any combination thereof. Herein, a skin- or membrane-like film (e.g., extruded or otherwise formed from polymer resin(s)) is not deemed a nonwoven. Herein, a paper tissue product, paper product, or paperboard or cardboard product, formed via wetlaying and predominantly constituted of tree wood pulp, is not deemed a nonwoven.

“Opened configuration,” with respect to a tampon, means the configuration of the pledget prior to the time it is compressed and formed into a self-sustaining form during manufacture, or in the case of a finished product, after it is completely ejected from an applicator (if present) and/or allowed and/or caused by any suitable technique to open and substantially re-assume its pre-compression shape and size.

“Paper” or “Cardboard” means a material in web or sheet form, formed predominantly of cellulose fibers, for example, wood pulp fibers, which have been suspended in a slurry, which is then poured onto a moving mesh belt, drained of water, and subsequently dried over drying rollers, and in many examples, finished via calendering. In the resulting web or sheet product the cellulose fibers are interlaid and randomly oriented. The paper or cardboard herein may also be substantially void of synthetic materials as defined herein, such as fibers, films, and/or adhesives. It is also to be appreciated that paper or cardboard may be formed by pulp molding.

“Predominant,” and forms thereof, when used to characterize a quantity of a constituent present in a composition, means that a majority of the weight of the material is constituted by the constituent.

“Withdrawal cord” refers to any section of string, yarn, cord, ribbon, strip material or other flexible/pliable elongate structure typically (although not necessarily) formed of fibrous material, attached to and/or extending from a tampon pledget and trailing from its rearward end. A withdrawal cord of sufficient length may be provided with a tampon for the purpose of providing a relatively thin and flexible trailing member of sufficient length to allow for a portion thereof to trail and remain outside of the introitus following full insertion of the tampon, which the user may easily grasp and pull to withdraw the tampon from her body following a desired duration of use.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

With respect to a paper sheet or web product, when laid out flat on a horizontal planar surface, the “z direction” is a direction orthogonal to the horizontal planar surface and is the direction along which caliper or thickness of the sheet or web (prior to rolling into paper tube product) would be measured.

The present disclosure related to absorbent articles including a tampon, and more particularly, to a tampon having an outer member and an inner member such that the inner member is slidably engaged with the outer member. The outer member has an outer surface surrounding a longitudinal applicator axis. The outer member includes a barrel region and a grip region. The grip region includes greater than four circumferentially formed faces. These greater than four faces provide greater functionality and case of use by providing multiple areas for the user to grip and securely hold the tampon during use. Each of the outer member and the inner member may include one or more layers that are spirally or convolutely wound to form the outer member and the inner member. Each of these one or more layers may comprise, consist essentially of, or consist of cellulosic material. Stated another way, each of the one or more layers may be void of synthetic fibers and films. A carboard applicator, as referred to herein, is an applicator that includes one or more layers and each of the one or more layers comprise, consists essentially of, or consists of cellulosic material and is void of synthetic fibers and films. “Synthetic” means that the polymerization did not happen in nature or was not naturally occurring. Synthetic materials are those that are chemically modified such that the polymerization was not naturally occurring. Synthetic fibers, for example, include synthetic polymeric materials and bio-plastic polymers, such as polyethylene (PE), polyethylene terephthalate (PET), polylactic acid (PLA), polyhydroxyalkanoates, polybutylene succinate. Materials that are not synthetic, for example, include cotton, flax, hemp, rayon, and jute. Free of synthetic materials means that the article or component of the article is at least about 90% or at least 95% or at least about 98% by weight void of materials where the polymerization did not happen in nature or was not naturally occurring.

Thus, the tampon of the present disclosure may have a cardboard applicator that provides greater functionality and convenience, and a more appealing ergonomic and/or aesthetic experience.

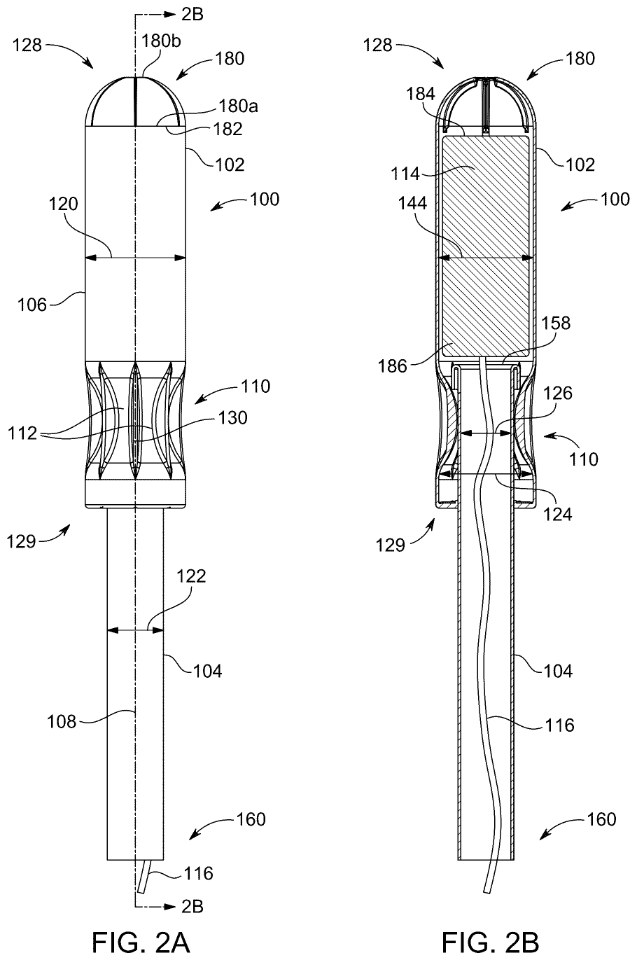

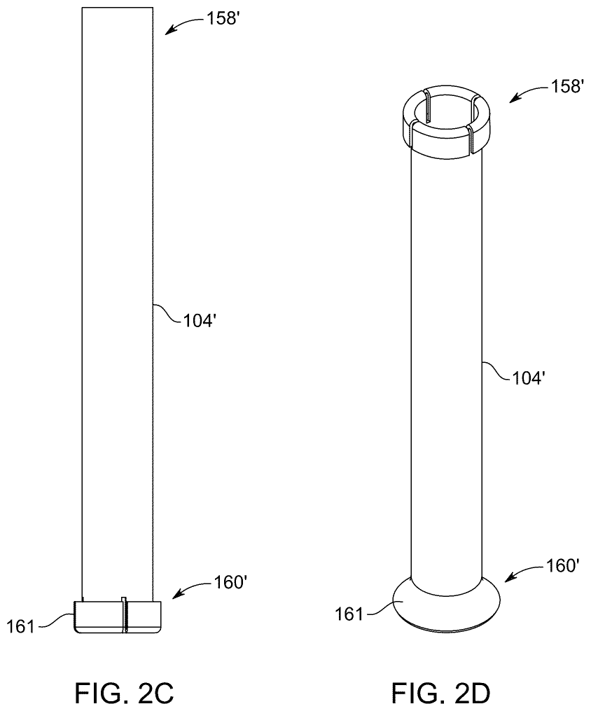

Referring to FIGS. 2A and 2B, a tampon in a self-sustaining form, including a pledget 114 having a forward end 184 and rearward end 186, and a withdrawal cord 116 attached to the pledget and trailing from the rearward end 186, may be supplied inside an applicator 100. Applicator 100 may be configured to house the tampon and protect it from contamination and/or unintended deformation prior to its use.

Applicator 100 may include an outer member including a hollow barrel region 102 and an ejection plunger 104 (also referred to herein as an inner member), which also may be hollow. The outer member, including the barrel region 102, and the ejection plunger 104 may be configured such that the ejection plunger 104 is at least partially disposed and longitudinally slidable within the barrel region 102. In some examples including the examples illustrated in FIGS. 2A and 2B, each of the barrel region 102 and the ejection plunger 104 are hollow approximately cylindrical bodies, wherein the cylindrical form of ejection plunger 104 has an external diameter 122 that is smaller than an inner diameter 144 of the barrel region 102, such that the barrel region 102 and the ejection plunger 104 may be concentrically arranged and longitudinally slidable with respect to each other in a telescoping manner. In some embodiments, the external diameter of the plunger or inner member is less than about 70% of the external diameter of the barrel.

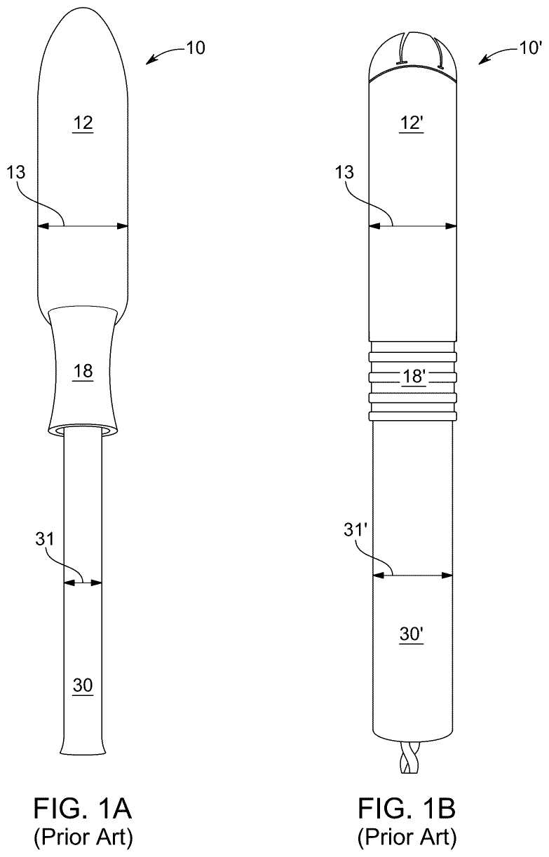

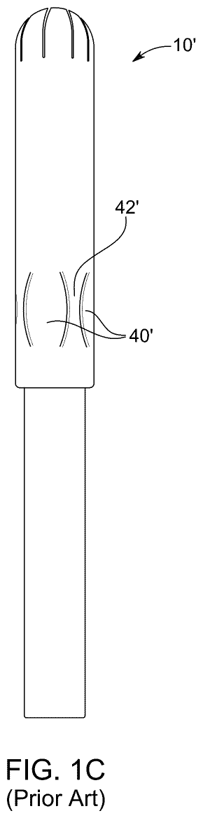

As shown in FIG. 2A, the outer member includes an outer surface 106 surrounding a longitudinal applicator axis 108, the barrel region 102, and the grip region 104. In some embodiments, the barrel region 102 has an external diameter 120 that is greater than the external diameter 122 of the plunger 104. In one example embodiment, the external diameter 120 of the barrel region is about 16 mm or in a range from about 12 mm to about 20 mm and the external diameter 122 of the plunger is about 7 mm or in a range from about 6 mm to about 8 mm. In some embodiments, the external diameter 122 of the plunger 104 is less than about 70% of the external diameter 120 of the barrel region 102. Unlike the conventional cardboard applicator 10′ (FIG. 1B) where the external diameter 31′ of the plunger 30′ is about 80-90% of the external diameter 13′ of the barrel region 12′, the external diameter 122 of the plunger 104 is about 50%-60% of the external diameter 120 of the barrel region 102. The inventors of the present disclosure recognized that the external diameter 31′ of the plunger 30′ in the conventional cardboard applicator 10′ had to be at least 80-90% of the external diameter 13′ of the barrel region 12′ to avoid the plunger 30′ wobbling as it is slid into or withdrawn from the barrel region 12′. The improved cardboard applicator 100 herein discloses an improved grip region 110 to accommodate the external diameter 122 of the plunger 104 only being 50-60% of the external diameter 120 of the barrel region 102 in order to prevent the undesired wobbling between the plunger 104 and barrel region 102.

The barrel region 102 may be provided with additional functional features. For example, a forward end 128 of the outer member may include an insertion end region adjacent to the barrel region. The insertion end region includes a plurality of petals 180 that have proximal bases 180a having outer surfaces coincident with the general cylindrical shape of the barrel region 102, and distal ends 180b disposed radially inwardly of the proximal portions. The forward end 128 of the barrel region 102 is opposite from the grip region 110. The petals 180 and/or remainder of the barrel region 102 may be configured such that the petals 180 may flex about the proximal bases 180a such that, upon forward longitudinal pressure exerted by the forward end 184 of the pledget 114 during ejection via pressure form longitudinal forward movement of the ejection plunger 104 relative to the barrel region 102, the petals 180 will flex outwardly about their bases. This expands an opening at the forward end 128 of the barrel region 102, which allows the pledget 114 to pass forwardly through the forward end 128 of the barrel region 102, and out of the applicator 100. The petals 180 may be integrally formed with the remainder of the barrel region 102. In some examples, the petals may be formed by making cuts in a forward portion of a cylindrical precursor body of the barrel region 102, and then plastically deforming the remaining uncut portions radially inwardly, and with suitable curvature. Such deformation may be accomplished in some examples via use of a forming die or mold (not shown), and may be facilitated in some examples via application of heat and/or steam. Additionally, in some examples the barrel region 102 may be imparted with localized flexure zones 182 that demark the proximal bases 180a of the petals, which are zones about which the material of the barrel region 102 and/or petals more readily flexes or hinges, for example, as a result of a localized reduction in thickness or caliper of the material in flexure zones 182. Flexure zones 182 may be imparted by, e.g., molding or localized compression applied during the manufacturing process.

The barrel region 102 may also be provided with a grip region 110 having gripping surface features (e.g., circumferential faces 112) that enable the user to tactilely identify a rearward end region 129 of the barrel region 102, and also enhance gripping slip resistance during use of the applicator 100. In a simple form as shown in FIGS. 2A and 2B, the features of the grip region 110 may be a series of circumferential faces 112 spaced apart by respective ribs 130 about the outer surface 106 of the barrel region 102 proximate its rearward end 129. More complex gripping surface features may be provided, however, of any design desired for purposes of functionality and esthetic appeal. Gripping surface features may be imparted to barrel region 102 via forming techniques including molding, embossing, etc.

As illustrated in FIG. 2B, the barrel region 102 may be sized to house the tampon pledget 114 in its self-sustaining form. It may be desired that the barrel region 102 have an internal diameter 144 that is small enough relative the outer lateral dimension of the pledget 114 such that the pledget 114 cannot freely slide longitudinally within the barrel region 102 (due to slight interference or radial compression and light static friction resistance), but not so small as to create unacceptable static friction resistance and thereby require the user to exert unacceptably great force on the ejection plunger or inner member 104 to eject the pledget 114 from the barrel region 102, when ejection is desired.

Also as suggested in FIG. 2A, it may be desired that the ejection plunger 104 have the external diameter 122 (of its cylindrical form) that is equal to or smaller than the internal diameter 144 or other inner lateral dimension of the barrel region 102. It may be desired to provide such clearance within 1% of parity such that the ejection plunger 104 cannot freely and unintentionally slide coaxially/longitudinally within or out of the barrel region 102 in the absence of intentionally applied force, but such that the force required to slide the ejection plunger 104 within the barrel region 102 is not so great as to be unacceptably great to the user, or even potentially destructive to the applicator.

Referring to FIGS. 2A and 2B, the ejection plunger 104 may be provided with an inner diameter that is smaller than an outer diameter or lateral dimension of the tampon pledget 114 (or other object) housed within the barrel region 102. With this size relationship, longitudinal force applied to the ejection plunger 104 urging it into the barrel region 102 will cause its forward end or first end region 158 to contact the rearward end 186 of the tampon pledget 114 (or other object) and urge the object forward and out the forward end 128 of the barrel region 102. As an alternative, or in addition to, such inner diameter size at the forward end region or first end region 158 of the ejection plunger 104 may have its circumferential edge rolled over inwardly or otherwise formed to extend radially inwardly, thereby effectively reducing the inner diameter or inner lateral dimension of the ejection plunger 104 at its forward end 158. The first end region 158 of the ejection plunger or inner member 104 may be outwardly rolled or flanged to increase the amount of area at the forward end 158 of the ejection plunger 104 that effectively contacts the rearward end 186 of the pledget 114 or other object within the barrel region 102, to transmit ejection force exerted by the user. A similar feature can be imparted to the rearward end or second end region 160 of the ejection plunger 104, to expand contact surface area and decrease focused/localized pressure on the user's finger at the circumferential rim of the rearward end 160 when the user applies ejection force at the rearward end 160, and thereby make application of ejection force more comfortable to the user's finger. In some embodiments, the pledget 114 is configured with an expulsion force of less than 900 gram-force (gf) or less than 800 gram-force (gf) or in a range of from about 400 to about 450 gf or from about 250 gf and about 700 gf.

In some embodiments, the barrel region 102 is manufactured such that the internal diameter 144 thereof is less than about 19 mm and preferably less than about 16 mm. Although the external diameter of typical menstrual-use tampons varies, most menstrual-use tampons currently marketed have an external diameter of less than about 19 mm. However, if one desires to use the applicator of the present disclosure for purposes other than delivery of a menstrual-use tampon to a human vaginal cavity (such as, for example, delivery of other devices, medications, etc. to other cavities in humans, or cavities in animals (veterinary use)), a barrel region 102 with a larger diameter may be desired.

The material of the outer member may be overlapped into a tubular configuration. Spirally or convolutely winding the outer member into a cylindrical tube is especially advantageous when the outer member is formed from a laminate. The reason for this is that when a laminate is circumferentially wound into a tube and a butt seam or an overlap is formed, a common problem with a rigid or stiff walled, tubular member having a relatively small diameter and a butt seam is that the seam may tend to come apart after formation if exposed to certain stress forces and/or high humidity. Accordingly, it may be preferred that the barrel region 102 be formed into a cylindrical configuration without the presence of a butt scam.

In some embodiments, the inner member 104 may be manufactured of similar materials and in a similar layered and wound configuration as the outer member, with appropriate adjustments to its size (length and outer diameter) to enable it to fit within the barrel region 102 with appropriate clearance, and have sufficient length to facilitate full ejection of the tampon pledget (or other object) from the barrel region 102.

For a menstrual-use tampon, the barrel region 102 may be sized and configured to snugly house the tampon. As noted above, it may be desired that the outer member have a relatively smooth exterior surface that will facilitate comfortable insertion of the barrel region 102 into the vaginal cavity. When the exterior surface is smooth (and preferably has surface properties and/or surface treatment that provide appropriately low friction with sensitive tissue), the barrel region 102 will easily slide within the vaginal cavity without subjecting the internal tissues of the vagina to irritation or abrasion. The exterior surface of the barrel region 102 may be coated to give it appropriate low friction characteristic surface properties. Wax, polyethylene, a combination of wax and polyethylene, and cellophane are representative components of coatings that have been applied to barrel regions of tampon applicators, to facilitate comfortable use.

The applicator of the present disclosure may be used for the delivery of menstrual-use devices, such as a tampon, an intravaginal collection device (e.g., menstrual cup), and interlabial pads. The applicator of the present disclosure may be used for the delivery of a pessary. The applicator may also be useful for delivery of oral, rectal, and vaginal suppositories, as well as nasal devices, such as nasal tampons. The applicator may be used for delivery of various other materials including, medicaments, moisturizers, vitamins and minerals, spermicides, and odor controlling agents. These materials may be delivered in the form of rigid solid objects, deformable solid objects, creams, foams, gels, etc. The applicator may be adapted for human or animal/veterinary use.

FIG. 2B depicts a non-limiting example of a tampon having the pledget 114 in a self-sustaining form and having a forward end 184 and a rearward end 186 and a withdrawal cord 116 attached to the pledget 114 and extending rearward from a location proximate the rearward end 186 of the pledget 114. Generally, the “pledget” as referred to herein is that portion of the tampon that includes the main body and mass of absorbent material, but does not include withdrawal cord 116. Tampons contemplated herein, however, are not limited to structures having the particular configuration shown in the drawing. In some embodiments, at least a portion of the cord 116 is braided. In other embodiments, there may be two different portions of the cord 116 that are each braided. In this example embodiment, one of the braided portions is a thick portion and the other of the braided portions is a thinner portion, as disclosed in U.S. Patent Publication No. 2023/0058874 that is incorporated by reference herein.

As shown in FIG. 2B, the pledget 114 of the tampon has the forward end 184 and the rearward end 186. During manufacture of the tampons the pledget 114 may be folded, bunched, compressed and/or otherwise formed in size and shape, from its initially manufactured configuration into a generally cylindrical and/or capsule-shaped configuration (e.g., as shown in FIG. 2B) along a radial direction, the lateral direction, longitudinal direction, or in some combination thereof.

FIG. 2C is a longitudinal side view of a plunger 104′ of the tampon product of FIG. 2A with a flange 161 at a second end region 160′ thereof. During use of the applicator 100, the plunger 104′ includes the forward end 158 and the rearward end 160 opposite to the forward end 158. The forward end 158 of the plunger 104′ is configured to engage a portion of the pledget 114 and the rearward end 160′ includes the flange 161. In some embodiments, the flange 161 can have a diameter greater than the internal diameter of the barrel region 102 at the rearward end 129 or the flange can have a diameter greater than a portion of the internal diameter of the grip region 110, to keep the plunger 104′ from unintentionally going through the barrel region 102 and out the forward end of the barrel, and to provide a surface on which the consumer or user can push to expel the pledget 114 from the forward end 128 of the barrel region 102, such as shown on FIG. 2D. In some embodiments, the flange 161 may be a pushed-out petal, a compressed portion, an embossed portion, or added material to increase the diameter of the plunger 104′ at the rearward end 160′. It is also to be appreciated that each of the first end region and/or the second end region may include a portion that has been rolled inwardly or outwardly or flanged.

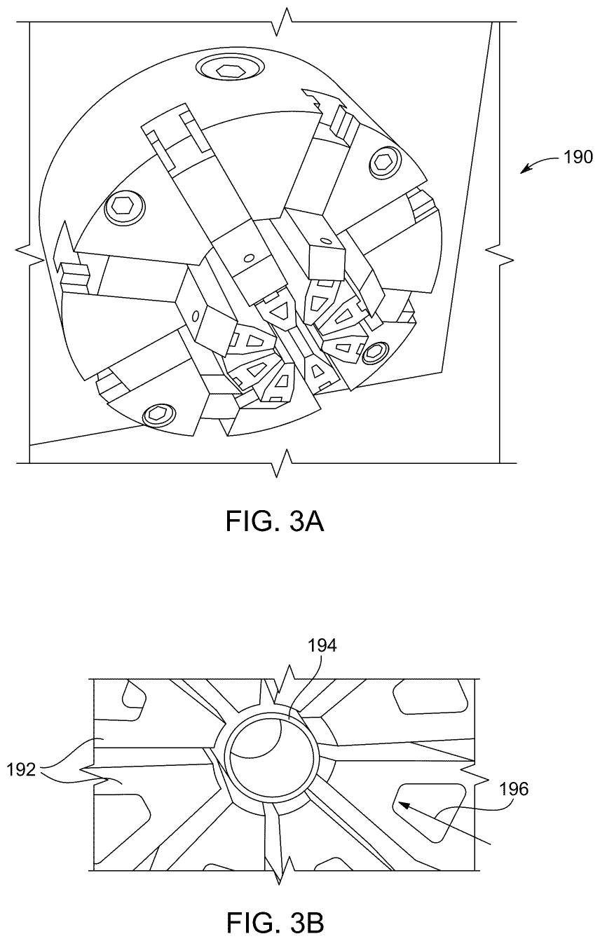

FIG. 3A is a perspective view of a compression device 190 used to form circumferential faces 112 in the grip region 110 of the applicator 100 of FIG. 2A. FIG. 3B is a perspective view of a plurality of forming members 192 of the machine 190 of FIG. 3A that move radially inward to form the circumferential faces 112 in the grip region 110 of the applicator 100. As shown in FIG. 3B to form the grip region 110 in the applicator 100, a tube 194, which may be a cardboard or paper tube, is positioned within an opening of the compression device 190. In some embodiments, the tube 194 is a spirally wound group of one or more layers, at least one of which includes cellulosic material. In other embodiments, the tube 194 is convolutely wound layers of material including at least one layer of cellulosic material.

In use, after positioning the cardboard tube 194 in the opening defined by the compression device 190, the forming members 192 are moved in an inward radial direction 196 and engage the outer surface of the tube 194 to form the circumferential faces 112 and ribs 130 therebetween. After forming the circumferential faces 112, the forming members 192 are moved in an opposite radial direction. The tube 194 is then moved or advanced such that a different section of the tube 194 is positioned within the opening and the process is repeated so that the grip region 110 including the circumferential faces 112 are formed at a plurality of spaced apart regions along the tube 194. The tube 194 is then removed from the compression device 190 and cut into individual parts where each part is used to form the barrel region 102 of the applicator 100. In other embodiments, the tube 194 may be first cut into a plurality of parts and each respective part is positioned in the opening of the compression device 190 and the forming members 192 are used to form the circumferential faces 112 in each part of the tube 194. The forming members 192 may be used to form the circumferential faces 112 without using a mandrel, a spindle, or any supporting material or device positioned inside the tube 194.

The inventors of the present invention recognized that the compression device 190 should be operated such that the paper or cardboard tube 194 is compressed to form the circumferential faces 112 without forming slits or holes in the tube 194. The inventors of the present disclosure surprisingly found that the tube 194 material may be compressed to form the circumferential faces 112 without affecting the color of the paper or cardboard tube 194. In some example embodiments, the forming members 192 of the compression device 190 radially compress the tube 194 material and pinch the material between two adjacent forming members 192 to form the circumferential faces 112 with ribs 130 therebetween. In some embodiments, heat is used in conjunction with the compression and squeezing of the forming members 192 but in other embodiments no heat is employed. The inventors of the present disclosure noted that there is some rebound to the cardboard tube 194 material as it is compressed to form the circumferential faces 112 and ribs 130.

The inventors of the present disclosure noted that for the conventional cardboard applicator 10′ (FIG. 1C), the greatest number of circumferential faces formed in the cardboard material is four. Thus, the inventors of the present disclosure herein configured the machine 190 to form greater than four circumferential faces 112 in the grip region 110. This is due to a larger number of circumferential faces in the grip region enhancing the control of the user over the barrel region 102 and/or the plunger 104. Unlike plastic material, which is relatively easy to mold and shape, the inventors of the present disclosure recognized that cardboard material is more challenging to mold and shape and thus had to overcome design obstacles not present in forming plastic applicators. For example, paper or carboard is prone to tearing when too much pressure is applied, and paper or cardboard tends to fold and produce wrinkles that would likely be aesthetically displeasing to consumers.

As previously discussed, conventional cardboard applicators 10′ (FIG. 1B) feature the barrel region 12′ with the external barrel diameter 13′ that is relatively close to the external plunger diameter 31′ of the plunger 30′. The inventors of the present disclosure expected that the compression and pinching of the paper or cardboard tube 194 with the forming members 192 would cause undesirable fold lines in the tube 194. Instead, the inventors of the present disclosure surprisingly found that the tube 194 tolerates the compression and squeezing of the forming members 192 relatively well, resulting in a surprisingly smooth surface to the circumferential faces 112 and ribs 130. Additionally, the inventors of the present disclosure expected that the compression and pinching of the tube 194 by the forming members 192 would require an additional heating step to form the circumferential faces 112 and ribs 130. However, the inventors surprisingly found that no additional heating step was required.

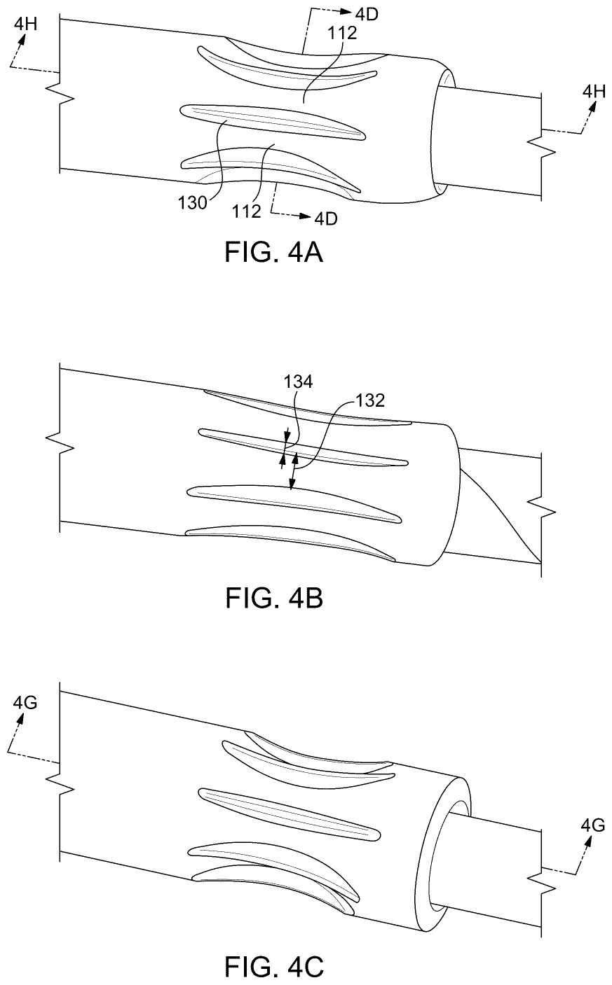

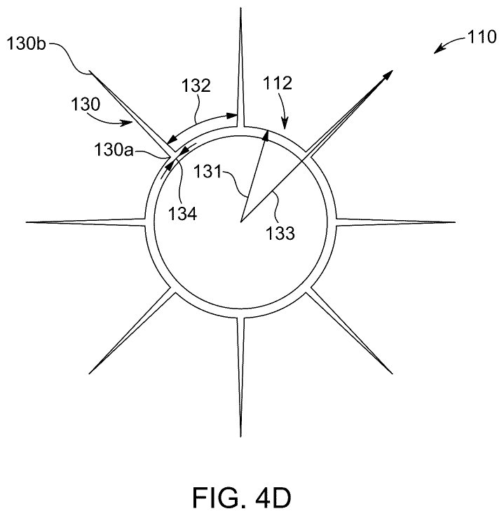

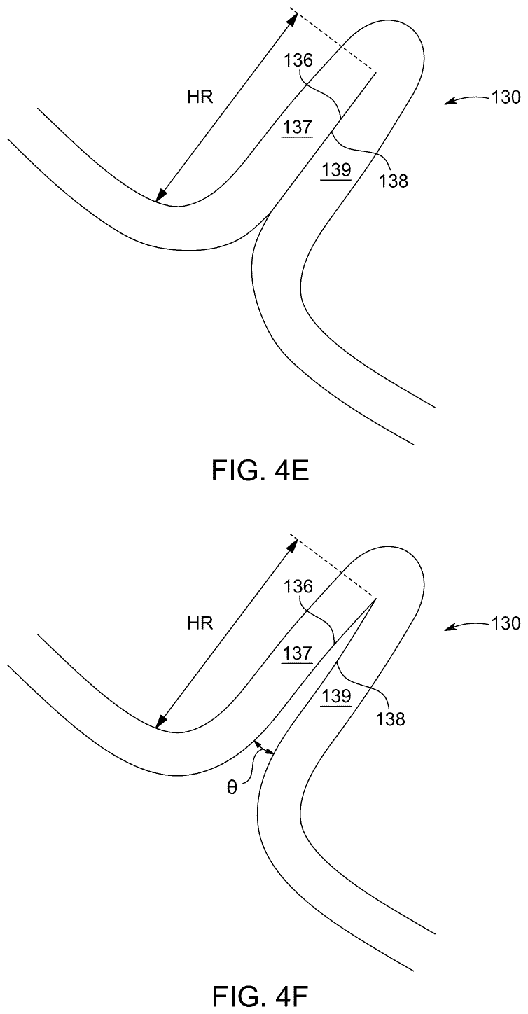

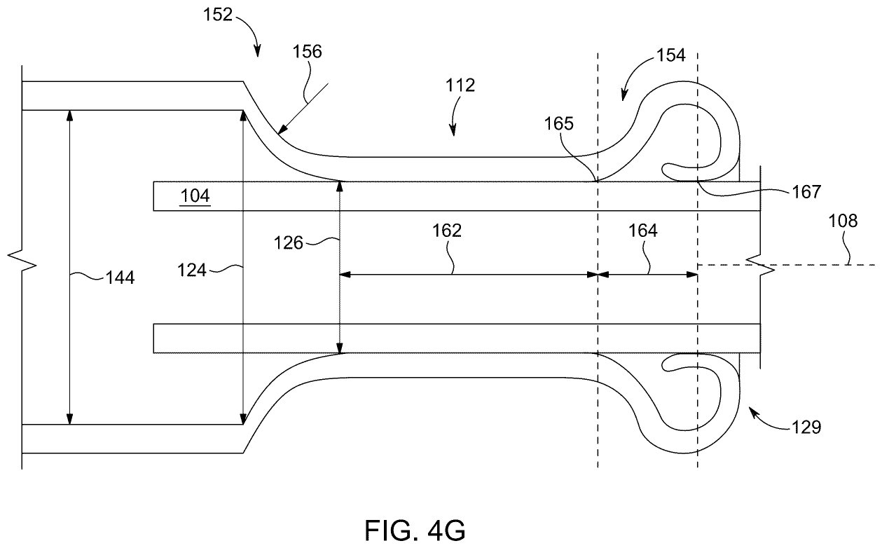

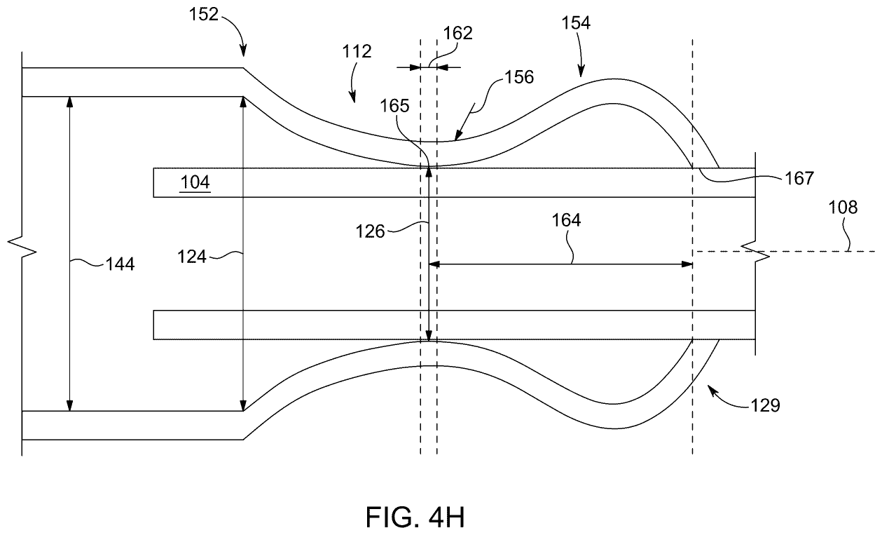

FIGS. 4A through 4C are perspective and top views of a plurality of circumferential faces 112 and ribs 130 therebetween of the grip region 110 of the applicator of FIG. 2A. In some embodiments, the circumferential faces 112 and ribs 130 therebetween are formed using the compression device 190 of FIGS. 3A and 3B. The circumferential faces may extend from an upper grip region to a lower grip region, as shown in FIGS. 4A-4C. Each of the faces 112 of the grip region 110, at least over a portion of the grip region, may be at least one of concave and convex. For example, two or more of the faces 112 may include a first concave region, a substantially planar region, and a second concave region. In another example, two or more of the faces 112 may be at least one of concave and convex extending from the upper grip region to the lower grip region. Each of the faces 112 may be identical in terms of shape and/or dimension. Each face 112 may have one or more of a same width (measured perpendicular to the longitudinal applicator axis 108), a same length (measured along the longitudinal applicator axis 108) and a same shape (e.g., same convex structure). In these embodiments, when operating the compression device 190 of FIGS. 3A and 3B, the compression by the forming members 192 is symmetrical in the inner radial direction 196 and/or each chuck 192 has a same shape or curvature to thereby compress a same-shaped circumferential face 112. In some embodiments, if an odd number of circumferential faces 112 are to be formed in the cardboard tube 194 then the compression force of the forming members 192 are to be symmetrical. In some embodiments, if an even number of circumferential faces 112 are to be formed in the cardboard tube 194 then faces which are radially opposite from each other have the same shape (e.g., be compressed by forming members 192 having a same shape).

As further shown in FIGS. 4A through 4C, in some embodiments the grip region 110 includes greater than four circumferentially formed faces 114. In other embodiments, the grip region 110 includes greater than or equal to six circumferential faces 114. In still other embodiments, the grip region 110 includes greater than or equal to eight circumferentially formed faces 114. As further shown in FIGS. 4A through 4C, the grip region 110 is continuously circumferential so that there are no slits or openings in the grip region 110. As further shown in FIGS. 4A through 4C, in some embodiments the grip region 110 comprises greater than four outwardly projecting ribs 130, where each rib 130 is positioned between two adjacent circumferential faces 112. The inventors of the present disclosure recognized that an increased number of circumferential faces enhance the user ability to grip the grip region 110. The peak region of the rib may be substantially planar with the external surface of the barrel region, or the peak region of the rib may be concave and/or convex with respect to the external surface of the barrel region. If the peak region of the rib is convex, the convexity of the peak region of the rib is less than the convexity of the face. Stated another way, the radius of curvature of the peak region of the rib is different and/or greater than the radius of curvature of the face.

In some embodiments, the circumferential faces 112 of the grip region 110 include one or more textures. Such textures feature embossed designs that may be formed through the same process used to form the circumferential faces using the compression device or in a subsequent or proceeding separate process. In still other example embodiments, additional material may be included outside of the grip region 110. Such additional material may include printed material (e.g., graphics, alphanumeric characters, etc.) or a material additive to create texture in the grip region 110.

FIG. 4D is a cross-sectional view of the grip region 110 of FIG. 4A taken along the line 4D-4D. As shown in FIGS. 4B and 4D, in some embodiments each of the circumferential faces 112 has a face circumferential width 132 and each of the ribs 130 has a rib circumferential width 134. In some embodiments, the face circumferential width 132 is greater than the rib circumferential width 134. In some embodiments, each of the faces 112 has the same face circumferential width 132 and each of the ribs 130 has the same rib circumferential width 134. The rib circumferential width 134 is measured at a rib base region 130a of the rib 130 (e.g., where the rib 130 intersects the face 112). The face circumferential width 132 is measured adjacent the face 112 surface. In some embodiments, the face circumferential width 132 is about 2 mm or in a range from about 0.03 mm to about 4.1 mm and the rib circumferential width 134 is about 0.6 mm or in a range from about 0.4 mm to about 0.8 mm. Thus, in these embodiments, a ratio of the face circumferential width 132 to the rib circumferential width 134 is about 3.0 or about 4.0 or in a range from about 0.1 to about 10.0 or from about 2.0 to about 10.0 or from about 3.0 to about 10.0. Thus, this design of the grip region 110 is distinct from the conventional cardboard applicator 10′ (FIG. 1C) with about a 1:1 ratio between the face circumferential width and the rib circumferential width. The inventors recognized that this alternate ratio in the disclosed cardboard applicator 100 enhances the grip of the user on the grip region and thus advantageously enhances the user control of the applicator during use. The rib circumferential width 134 may be variable from the rib base region 130a to the rib peak region 130b. Stated another way, the rib 130 may be tapered from the rib base region 130a to the rib peak region 130b such that the rib base region 130a has a greater width than the rib peak region 130b. In some embodiments, the rib circumferential width 134 may be substantially the same in the rib peak region 130b and the rib base region 130a.

In some other embodiments, such as shown in FIG. 4D each of the circumferential faces 112 has an external face radius 131 and each of the ribs 130 has external rib radius 133. Along at least a portion of the face, the external rib radius 133 is greater than the external face radius 131. Each of the faces 112 may have the same external face radius 131 and each of the ribs 130 may have the same external rib radius 133. In some embodiments, the external rib radius 133 is about 7.0 mm or in a range from about 6.0 mm to about 10.0 mm and the external face radius 131 is about 3.5 mm or in a range from about 3.0 mm to about 7.0 mm. Thus, in these embodiments, a ratio of the external rib radius 133 to the external face radius 131 is about 2.0 or in a range from about 0.8 to about 3.0. Thus, this is distinct from the conventional cardboard applicator 10′ (FIG. 1C) where the ratio is much smaller which reduces the ability of the user to engage and grip the grip region of the applicator. The inventors recognized that this larger ratio in the disclosed cardboard applicator 100 enhances the grip of the user on the grip region (e.g., by providing sufficient radial space to position a user's finger so to grip one or more ribs 130) and thus advantageously enhances the user control of the applicator during use. In some embodiments, a distance between the peak region 130b of a first rib 130 and the peak region 130b of a second rib 130 that is radially opposite of the first rib 130 is about 17 mm or in a range from about 15 mm to about 20 mm.

FIG. 4E is a cross-sectional view of a rib of the cross-sectional view of the grip region 110 of FIG. 4A. As shown in FIG. 4E, in some embodiments, the rib 130 includes a first rib portion 137 and a second rib portion 139 that are compressed or squeezed together (e.g., by the forming members 192 of the compression device 190) to form the rib 130. In some embodiments, a first internal rib surface 136 of the first rib portion 137 is in contact with a second internal rib surface 138 of the second rib portion 137. In some embodiments, the first internal rib surface 136 contacts the second internal rib surface 138 over at least a portion (e.g., greater than about 5% or greater than about 10% or greater than about 15%) of a rib height RH of the rib 130. In some embodiments, the first internal rib surface 136 contacts the second internal rib surface 138 over at least a majority (e.g., greater than 50%) of a height RH of the rib 130. In other embodiments, the first internal rib surface 136 contacts the second internal rib surface 138 over the entire height RH of the rib 130. The inventors of the present disclosure recognized that having these internal rib surfaces 136, 138 contact each other over at least a portion of the rib 130 height maximizes the face circumferential width 132 and/or minimizes the rib circumferential width 134. Consequently, as previously discussed, this advantageously enhances the user control over the grip region 110 by enhancing the ability of the user to grip the barrel region 102.

FIG. 4F is a cross-sectional view of a rib of the cross-sectional view of the grip region 110. As shown in FIG. 4F, in some embodiments, the rib 130 includes a first rib portion 137 and a second rib portion 139 that are compressed or squeezed together (e.g., by the forming members 192 of the compression device 190) to form the rib 130. In some embodiments, a first internal rib surface 136 of the first rib portion 137 is in separated from a second internal rib surface 138 of the second rib portion 137 by a rib angle Θ. The rib angle Θ may be from about 0 degrees to about 15 degrees or from about 0 degrees to about 10 degrees, including all 0.1 degree increments form therein and thereby the recited range. The inventors of the present disclosure recognized that minimizing the distance between the internal rib surfaces 136, 138 maximizes the face circumferential width 132 and/or minimizes the rib circumferential width 134. Stated another way, the inventors of the present disclosure recognized that angling the internal rib surfaces 136, 138 with respect to one another maximizes the face circumferential width 132 and/or minimizes the rib circumferential width 134. Consequently, as previously discussed, this advantageously enhances the user control over the grip region 110 by enhancing the ability of the user to grip the barrel region 102. The amount of rebound or separation between the first internal rib surface and the second internal rib surface will be dependent on the aggressiveness of the amount of compression or the intended design, such as the ratio of rib radius to rib face radius. The larger the difference between these radii, the more likely there is to be a gap since the stress is concentrated on the tips of the rib and the fold at the base of the rib, causing the rib base to separate.

FIG. 4G is a cross-sectional view of the grip region 110 of FIG. 4B taken along the line 4G-4G. As shown in FIG. 4G, in some embodiments each of the circumferential faces 112 of the grip region 110 project inwardly toward the longitudinal applicator axis 108.

As further shown in FIG. 4G, the grip region 110 has a first internal grip diameter 124 and a second internal grip diameter 126, where the first internal grip diameter 124 is greater than the second internal grip diameter 126. Thus, in these embodiments, the grip region 110 features different diameters at different portions along the longitudinal applicator axis 108. In some embodiments, the first internal grip diameter 124 is about 13.3 mm or in a range from about 9.0 mm to about 20.0 mm and the second internal grip diameter 126 is about 6.0 mm or in a range from about 5.0 mm to about 14.0 mm. In some embodiments, a ratio of the second internal grip diameter 126 to the first internal grip diameter 124 is less than about 90%. The difference between the first internal grip diameter and the second internal grip diameter may be at least about 1 mm or at least about 1.5 mm or at least about 2 mm or at least about 2.5 mm or at least about 3 mm or at least about 3.5 mm or at least about 4 mm or at least about 5 mm or from about 1.5 mm to about 13 mm or from about 2.5 mm to about 10 mm. Thus, the circumferential faces 112 extend within the interior of the barrel region 102 towards the longitudinal applicator axis 108 by a greater extent than the faces 40′ in the conventional cardboard applicator 10′ (FIG. 1C). The inventors of the present disclosure recognized that this advantageously ensures tight engagement between the plunger 104 and the grip region 110. Since the disclosed applicator 100 herein features the external plunger diameter 122 being noticeably less than the external barrel diameter 120 (FIG. 2A). The external plunger diameter 122 is measured (or defined) where the flange 161 or marking in regions 170, 172, 174 are not present. The inventors of the present disclosure recognized that this ratio between the second internal grip diameter 126 and first internal grip diameter 124 facilitates tight engagement between the plunger 104 and the barrel region 102. In some embodiments, the second internal grip diameter 126 is at least 30% of the first internal grip diameter 124. In some embodiments, a difference between the first internal grip diameter 124 and the second internal grip diameter 126 is at least about 1 mm and/or about 2.5 mm and/or about 3 mm and/or about 3.5 mm and/or about 4 mm or from about 1 mm to about 15 mm.

The inventors of the present disclosure designed the grip region 110 so that the barrel region 102 may support the plunger 104 at multiple regions along the longitudinal applicator axis 108. As shown in FIG. 4G, in some embodiments the plunger 104 is supported by the grip region 110 (at the second internal grip diameter 126) and is also supported at the rearward end 129 of the barrel region 102. As illustrated in FIGS. 4C and 4G, the rearward end region 129 of may be inverted or rolled such that an external surface of the rearward end is configured to become an internal surface and engage a portion of the plunger 104. The external plunger diameter 122 of the plunger 104 (FIG. 2A) is greater than 90% of the second internal grip diameter 126 of the grip region 110. The inventors of the present disclosure recognized that this facilitates tight engagement between the plunger 104 and the grip region 110 while still permitting the plunger 104 to slide within the barrel region 102 relative to the grip region 110. For purposes of this disclosure, “tight engagement” corresponds to a tolerance (e.g., difference between the external plunger diameter 122 and the second internal grip diameter 126) that is about 0.3 mm to about 0.5 mm and/or about 0.2 mm to about 0.6 mm and/or about 0.1 mm to about 0.8 mm.

As shown in FIG. 4G, in some embodiments the second internal grip diameter 126 of the grip region 110 contacts the plunger 104 over a minimum longitudinal length 162 along the longitudinal applicator axis 108. The minimum longitudinal length 162 may be at least about 12 mm or at least about 15 mm or in a range from about 12 mm to about 20 mm along the longitudinal applicator axis 108. As shown in FIG. 4G in some embodiments the circumferential faces 112 of the grip region 110 are substantially flat over the minimum longitudinal length 162.

As further shown in FIG. 4G, in some embodiments the grip region 110 of the barrel region 102 contacts the plunger 104 at a first contact point 165 and the rearward end 129 of the barrel region 102 contacts the plunger 104 at a second contact point 167. The contact points 165, 167 are spaced apart along the longitudinal applicator axis 108 by a distance 164. The distance 164 is about 8 mm or in a range from about 6 mm to about 10 mm. The inventors of the present disclosure recognized that providing multiple contact points 165, 167 between the plunger 104 and the barrel region 102 advantageously helps to stabilize the plunger 104 as it is inserted into and retracted from the barrel region 102. The inventors of the present disclosure also recognized that spacing the contact points 165, 167 by a distance disclosed herein further facilitates this stabilization.

As further shown in FIG. 4G, in some embodiments each of the circumferential faces 112 extends from an upper grip region 152 to a lower grip region 154. In some embodiments, the faces 112 have a radius of curvature 156 in one or both of the regions 152, 154 that is about 8.3 mm or in a range from about 0.1 mm to about 425 mm. The radius of curvature may be determined by taking the (face length)²/(face depth). The face length may be from about 3 m to about 30 mm and the face depth may be from about 0.1 mm to about 3 mm. For example, the face length may be about 25 mm and the face depth may be about 2 mm. In some embodiments, the radii of curvature is the same for each of the circumferential faces 112. In this example embodiment, the radius of curvature 156 is measured in a plane perpendicular to the longitudinal applicator axis 108. The inventors of the present disclosure recognized that having the radius of curvature 156 in this range advantageously maximizes the length of the circumferential faces 112 (e.g., along the longitudinal applicator axis 108) which further enhances the user ability to grip the grip region 110 and thus maximizes user control of the barrel region 102. Although FIG. 4G depicts that the grip region 110 is substantially cylindrical between the regions 152, 154 in other embodiments the grip region 110 is concave or convex between the regions 152, 154.

It is also to be appreciated that the rearward end region may not contact the plunger. The rearward end region may be shaped such that it extends outward away from the plunger or stated another way, there is a gap between the plunger and the rearward end region. In those embodiments where the reward end region does not contact the plunger, the face may engage or contact the plunger along the minimum longitudinal length, as previously discussed.

As illustrated in FIG. 4H, in some embodiments the plunger 104 is supported by the grip region 110 (at the second internal grip diameter 126) and is also supported at the rearward end 129 of the outer member. As illustrated in FIGS. 4A and 4H, the rearward end region 129 of may be configured to engage a portion of the plunger 104. The external plunger diameter 122 of the plunger 104 (FIG. 2A) is greater than 90% of the second internal grip diameter 126 of the grip region 110. The inventors of the present disclosure recognized that this facilitates tight engagement between the plunger 104 and the grip region 110 while still permitting the plunger 104 to slide within the barrel region 102 relative to the grip region 110.

As shown in FIG. 4H, the second internal grip diameter 126 of the grip region 110 contacts the plunger 104 over a minimum longitudinal length 162 along the longitudinal applicator axis 108. The minimum longitudinal length 162 may be at least about 2 mm or at least about 5 mm or at least about 7 mm or in a range from about 2 mm to about 10 mm along the longitudinal applicator axis 108. As shown in FIG. 4H, in some embodiments, the circumferential faces 112 of the grip region 110 are substantially curved over the minimum longitudinal length 162.

As further shown in FIG. 4G, in some embodiments the grip region 110 of the barrel region 102 contacts the plunger 104 at a first contact point 165 and the rearward end 129 of the barrel region 102 contacts the plunger 104 at a second contact point 167. The contact points 165, 167 are spaced apart along the longitudinal applicator axis 108 by a distance 164. The distance 164 is about 8 mm or in a range from about 6 mm to about 10 mm. The inventors of the present disclosure recognized that providing multiple contact points 165, 167 between the plunger 104 and the barrel region 102 advantageously helps to stabilize the plunger 104 as it is inserted into and retracted from the barrel region 102. The inventors of the present disclosure also recognized that spacing the contact points 165, 167 by a distance disclosed herein further facilitates this stabilization.

As further shown in FIG. 4H, each of the circumferential faces 112 extends from an upper grip region 152 to a lower grip region 154. In some embodiments, the faces 112 have a radius of curvature 156 in one or both of the regions 152, 154 that is about 300 mm or in a range from about 4.3 mm to about 900 mm. The radius of curvature is determined by taking the (face length) 2/(face depth). The face length may be from about 3 m to about 30 mm and the face depth may be from about 0.1 mm to about 3 mm. For example, the face length may be about 25 mm and the face depth may be about 2 mm. In some embodiments, the radii of curvature is the same for each of the circumferential faces 112. The radius of curvature 156 is measured in a plane perpendicular to the longitudinal applicator axis 108. The inventors of the present disclosure recognized that having the radius of curvature 156 in this range advantageously maximizes the length of the circumferential faces 112 (e.g., along the longitudinal applicator axis 108) which further enhances the user ability to grip the grip region 110 and thus maximizes user control of the barrel region 102. Although FIG. 4G depicts that the grip region 110 is substantially concave between the regions 152, 154, the grip region 110 be substantially flat, concave, and/or convex between the regions 152, 154.

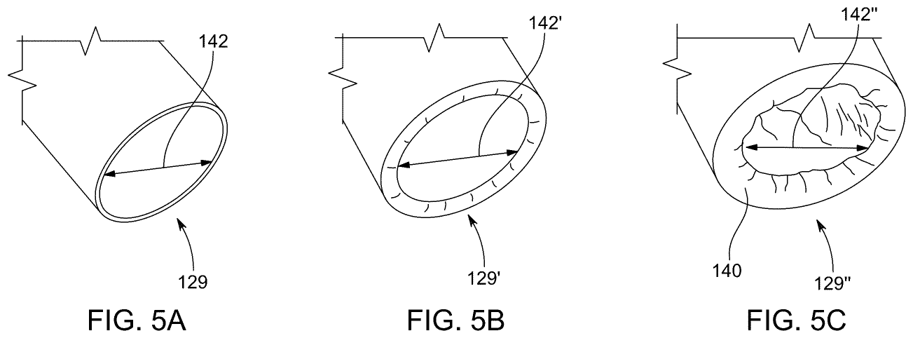

FIGS. 5A through 5C are perspective views of the rearward end 129 of the barrel region 102 of the applicator 100 of FIG. 2A. As shown in FIGS. 5A through 5C, the barrel region 102 includes a rearward end 129 disposed adjacent to the grip region 110. The rearward end 129 comprises an internal end region diameter 142. FIG. 5A shows the rearward end 129 with the internal end region diameter 142 without any modification to the rearward end 129. FIGS. 5B and 5C depict respective rearward ends 129′, 129″ where the rearward end has been modified (e.g., by rolling) so to reduce the internal end region diameter 142′, 142″ relative to the internal end region diameter 142 in FIG. 5A. In these embodiments, the internal end region diameters 142′, 142″ are each less than the internal barrel diameter 144. In some embodiments, where the second contact point 167 of FIGS. 4G and 4H is between the plunger 104 and the rearward end 129″ of FIG. 5C with the internal end region diameter 142″, the external plunger diameter 122 is at least 90% of the internal end region diameter 142″. This facilitates the same tight engagement at the second contact point 167 as was discussed previously with respect to the first contact point 165. In some embodiments, the rearward end 129″ of FIG. 5C includes a rolled edge 140 that has the internal end region diameter 142″. The difference between the rearward end 129′ of FIG. 5B and rearward end 129″ of FIG. 5C is the degree to which the rearward end has been rolled. FIG. 5C is rolled to a greater extent in the rearward end 129″. The inventors of the present disclosure recognized that the rolled edge 140 advantageously results in a smaller internal end region diameter 142″ which can aid in stabilizing the plunger 104 particularly at the rearward end 129 of the barrel region 102. Without the rolled edge 140 of the rearward end 129″, the plunger 104 would only contact the interior surface of the barrel region 102 at the first contact point 165 and thus there would not be as much stability between the plunger 104 and barrel region 102 as the plunger 104 is moved into and out of the outer member.

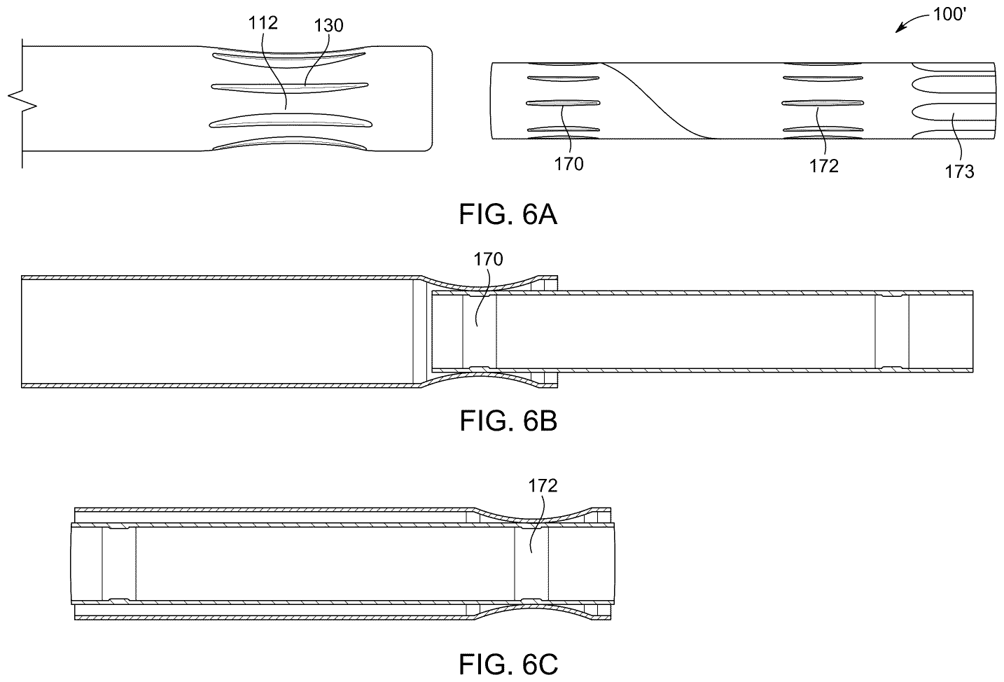

FIGS. 6A through 6C are side views of an applicator 100′ including a barrel region and a plunger with circumferential faces formed in both the barrel region and the plunger. As illustrated in FIG. 6A, the plunger 104 features circumferential faces and/or other markings formed in multiple regions 170, 172, 173. The circumferential faces formed in the multiple regions 170, 172, 173 of the plunger 104 provide physical faces that may cause the plunger 104 to advance differently as the plunger is inserted into the barrel region 102. For example, these multiple regions 170, 172, 173 may result in areas of increased or decreased friction as the plunger advances into the barrel. However, these regions of the plunger 104 are moderate and can be easily overcome by the user applying additional force or less force to the plunger 104. In some embodiments, such as in FIG. 6B, the plunger 104 inserted within the barrel region 102 until the circumferential faces in the first region 170 engage the grip region 110 of the barrel region 102. FIG. 6C then depicts the plunger 104 after being inserted further within the barrel region 102 until the circumferential faces in the second region 172 engage the grip region 110 and the circumferential faces in the third region 173 are positioned at the end region 129 of the barrel region 102. The circumferential faces (or markings) formed in the multiple regions 170, 172, 173 of the plunger 104 assist the user when inserting the plunger 104 into the barrel region 102 by providing either physical indicators (circumferential faces) or visual indicators (markings) when the plunger 104 has reached various phases of insertion within the barrel region 102. In the embodiments where circumferential faces are formed in the multiple regions 170, 172, 173 of the plunger 104, these circumferential faces are formed in a similar manner (e.g., using the compression device 190 of FIGS. 3A and 3B) as the circumferential faces formed in the barrel region 102.

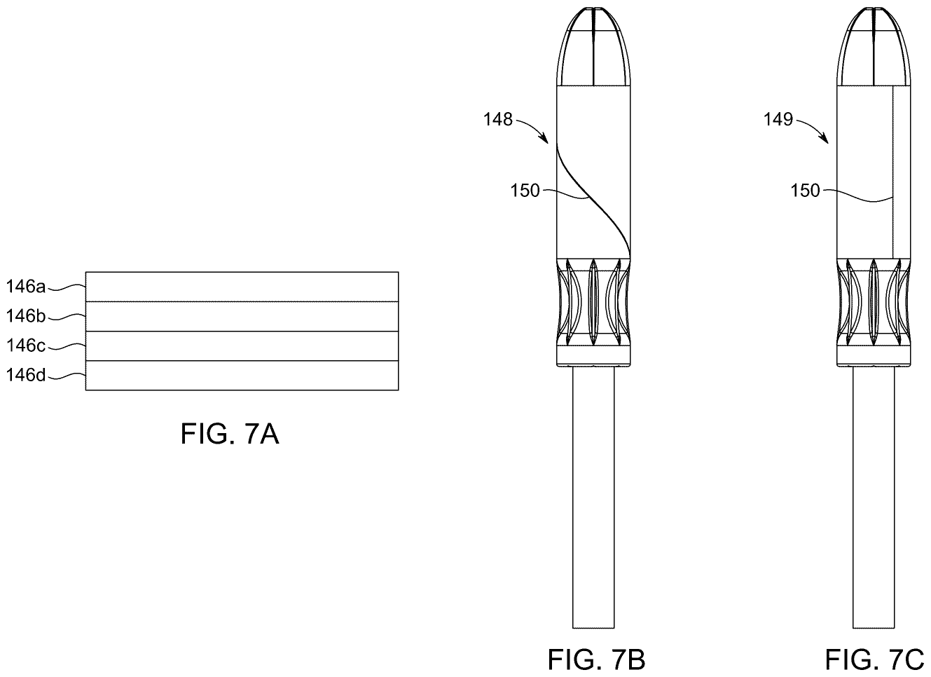

FIG. 7A is a cross-sectional side view of a plurality of material layers 146a through 146d that define the applicator 100. Although four layers 146a through 146d are depicted in FIG. 7A, in other embodiments less or more than four layers may define the applicator 100. The one or more of the layers 146a through 146d may exclude non-naturally occurring fibers. The one or more layers 146a through 146d may include naturally-occurring fibers. Naturally occurring fibers may include cellulosic fibers, wool, silk and other naturally-occurring protein fibers and mixtures thereof, cotton, cotton linters, flax, sisal, abaca, hemp, hesperaloe, jute, bamboo, bagasse, kudzu, corn, sorghum, gourd, agave, loofah, trichomes, seed-hairs, wheat, and mixtures thereof. In other embodiments, each of the one or more layers 146a through 146d include cellulosic material including wood fibers. Wood fiber(s) means fibers derived from both deciduous trees (hereinafter, also referred to as “hardwood”) and coniferous trees (hereinafter, also referred to as “softwood”) may be utilized. Wood fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. The one or more of the layers 146a through 146d may be free of synthetic fibers and films.

FIG. 7B is a side view of an applicator including the material layers 146a through 146d of FIG. 7A spirally wound to form the barrel region 102. In some embodiments, the one or more layers 146a through 146d are spirally wound to form at least one of the barrel region 102 and/or plunger 104. FIG. 7B depicts the spiral or non-linear seam 150 formed when the layers 146a through 146d are spirally wound to form the barrel region 102. In some embodiments, one or more of the layers 146a through 146d can include other materials, such as plastic. However, in other embodiments, each of the layers 146a through 146d are 100% cellulosic. In some embodiments, the cellulosic material of the one or more layers 146a through 146d is paper. In other embodiments, the cellulosic material of the one or more layers 146a through 146d is pulp.

FIG. 7C is a side view of an applicator including the material layers 146a through 146d of FIG. 7A convolutely wound to form the barrel region 102. In some embodiments, the one or more layers 146a through 146d are convolutely wound to form at least one of the barrel region 102 and/or plunger 104. FIG. 7C depicts the linear seam 150 formed when the layers 146a through 146d are convolutely wound to form the barrel region 102. In some embodiments, one or more of the layers 146a through 146d can include other materials, such as plastic. However, in other embodiments, each of the layers 146a through 146d are 100% cellulosic. In some embodiments, the cellulosic material of the one or more layers 146a through 146d is paper. In other embodiments, the cellulosic material of the one or more layers 146a through 146d is pulp.

The outer member may be provided in the form of a spirally wound, convolutely wound or other seamed hollow tube which may be formed from paper, paperboard, cardboard or a combination thereof. The paper, paperboard, or cardboard may be manufactured predominantly of wood and/or cellulose pulp fiber. The barrel region 102 may be manufactured to be relatively rigid and have an external barrel diameter 120 of about 10 millimeters to about 20 millimeters. The barrel region 102 has a wall which may be manufactured to have a predetermined thickness of about 0.1 mm to about 0.7 mm. The wall may be constructed from a single ply of material or be formed from two or more plies that are bonded together to form a laminate.

The use of two or more plies or layers enables the manufacturer to use particularly selected materials in the various layers that may enhance the performance of the tampon applicator 100. When two or more layers are included, all the layers may be spirally wound, convolutely wound, and/or longitudinally seamed to form an elongated cylinder. The wall may be constructed using a relatively smooth thin ply of material on the outside or exterior surface that surrounds a relatively coarser and possibly thicker ply. When the wall includes at least three layers, the middle ply may be the thicker ply and the interior and exterior layers may be imparted with a relatively smooth and/or slippery surface finish (e.g., via application of a coating) to facilitate expulsion of the tampon and to facilitate comfortable insertion of the barrel region 102 into the vaginal cavity, respectively. By sandwiching a thick, coarser ply of material between two thin, smooth layers, a barrel region 102 may be provided which is economical and functional. The wall may include one to three, four or five layers, although more layers may be utilized if desired.

The plies, or layers, forming the wall may be held together by a coating, such as an adhesive, or by a coating activated by heat and/or pressure, or by mechanical interaction such as by ultrasonic bonding or pressure bonding, or a combination thereof. The coating may be a petroleum, a petroleum derivative, and/or a plastic with petroleum or petroleum derived components or precursor materials. Thus, an applicator may comprise less than 10 percent by weight or less than 5 percent by weight petroleum, petroleum derivatives and/or plastics with petroleum or petroleum-derived components or precursor materials. For example, each of the layers of the applicator may comprise a cellulosic material, such as paper, and the coating between the layers or plies may be a petroleum, a petroleum derivative, and/or a plastic with petroleum or petroleum derived components or precursor materials. Alternatively, the applicator may comprise a coating, such as an adhesive that is not a petroleum, a petroleum derivative, and/or a plastic with petroleum or petroleum derived components or precursor materials.

The coating may be either water-soluble or water-insoluble. For example, a water-soluble coating, such as a water-soluble adhesive, may be preferred, for reasons of environment-related concerns, so that the layers of the wall will readily separate when wetted by water. Such wetting may occur, for example, upon immersion by flushing in a toilet, or upon exposure to environmental moisture (for example, in an outside landfill in moist or wet conditions). When a water-soluble coating is used, exposure of the barrel region 102 to processes of a municipal waste treatment plant, wherein soaking in water, interaction with chemicals and agitation all occur, will cause the layers of the wall to separate and disperse in a relatively short period of time.

In addition to the objective of providing for dissolution and dispersibility, as noted above, it may be desirable to reduce or eliminate components derived from petroleum according to currently recognized objectives relating to use of sustainably-sourced and environmentally-friendly materials. Many (if not all) adhesives currently used to adhere layers of paper together to form tube structures for applicator components are aqueous formulations including polyvinyl acetate (PVAc). PVAc-based adhesive (exemplified by “white glue” or ELMER'S brand glue) is desirable because it is water soluble, readily penetrates pores of fibrous cellulose/wood pulp paper and adheres to cellulose fibers, is tacky when applied (promoting rapid setting and bonding), and can be formulated with relatively high water content and low viscosity for case of application during manufacturing. However, PVAc is derived from hydrocarbons (acetylene or ethylene), most economically obtained from petroleum. Other non-petroleum-derived adhesive components might be considered, including hide glue and sodium silicate (water glass), used elsewhere in paper-related applications.

When tube stock used to form an applicator or ejection plunger is formed of paper (in turn, formed of cellulose pulp such as wood pulp), the paper may be relatively hydrophilic, porous and absorbent, and also, potentially abrasive to sensitive tissue prior to a coating being applied. The hydrophilic, porous and absorbent qualities may cause the paper material to draw moisture from moist tissues, and thereby increase the potential for irritation and abrasion from sliding contact therebetween. Additionally, these qualities may increase the potential for staining of the paper material with menstrual fluid, which, following withdrawal of an applicator from the vaginal cavity, some users may find undesirable. Accordingly, it may be desirable to apply a coating to the material that, when in melt and/or liquid form readily penetrates the fibrous structure of the paper and seals it to prevent it from absorbing fluid. The coating may be applied to one or more layers or plies, such as the outer layer of the barrel and/or the inner layer of the barrel. The coating may be applied to the layer or ply prior to forming the applicator. More specifically, the coating may be applied prior to the one or more layers being convolutely wound and/or spirally wound.

Heat and/or steam and/or moisture may be applied to the one or more layers and/or the cylindrical segment to aid in forming the grip region. However, the increased moisture content during manufacture of the outer member and/or the inner member does not remain after formation of the outer member/inner member. The tampon applicator, including at least one of the outer member and the inner member, of the present invention may have a water (moisture) content (% water in sample) from 0% to about 20%; in certain embodiments, tampon applicator of the present invention may have a water (moisture) content from about 1% to about 15%; in certain embodiments, tampon applicator of the present invention may have a water (moisture) content from about 2% to about 10%; and in certain embodiments, tampon applicator of the present invention may have a water (moisture) content from about 4% to about 10%, wherein the water content is measured by the Water Content Test Method described herein.

### Test Methods

### Water Content Test Method

The water (moisture) content present in a tampon applicator of the present invention is measured using the following Water Content Test Method. A tampon applicator or portion thereof (“sample”) is placed in a conditioned room at a temperature of 23° C.±1.0° C. and a relative humidity of 50%±2% for at least 24 hours prior to testing. Under the temperature and humidity conditions mentioned above, using a balance with at least four decimal places, the weight of the sample is recorded every five minutes until a change of less than 0.5% of previous weight is detected during a 10 minute period. The final weight is recorded as the “equilibrium weight”. Within 10 minutes, the samples are placed into the forced air oven on top of foil for 24 hours at 70° C.±2° C. at a relative humidity of 4%±2% for drying. After the 24 hours of drying, the sample is removed and weighed within 15 seconds. This weight is designated as the “dry weight” of the sample.

The water (moisture) content of the sample is calculated as follows:

$$\% Water in sample = 100 \% \times \frac{( Equilibrium weight of sample = Dry weight of sample )}{Dry weight of sample}$$

The % Water (moisture) in sample for 3 replicates is averaged to give the reported % Water (moisture) in sample. Report results to the nearest 0.1%.

### Combinations

The following combinations are contemplated herein:

A1. A tampon product comprising: an applicator comprising an outer member and an inner member slidably engaged with the outer member, the outer member comprising an outer surface surrounding a longitudinal applicator axis, a barrel region, and a grip region, wherein the grip region comprises greater than four circumferentially formed faces.

A2. The tampon product according to paragraph A1, comprising a tampon, wherein the tampon comprises a pledget and a cord attached to the pledget, wherein the pledget is disposed within at least a portion of the barrel region of the applicator.

A3. The tampon product according to any one of the preceding paragraphs, wherein the barrel region has an external barrel diameter and the inner member has an external inner member diameter, and wherein the external inner member diameter is less than 70% of the external barrel diameter.

A4. The tampon product according to any one of the preceding paragraphs, wherein the grip region has a first internal grip diameter and a second internal grip diameter, wherein the first internal grip diameter is greater than the second internal grip diameter.

A5. The tampon product according to paragraph A4, wherein the inner member has an external inner member diameter, and wherein the external inner member diameter is greater than 90% of the second internal grip diameter of the grip region.

A6. The tampon product according to paragraph A4, wherein the difference between the first internal grip diameter and the second internal grip diameter is at least about 2 mm.

A7. The tampon product according to any one of the preceding paragraphs, wherein the outer member comprises an insertion end region opposite the grip region.

A8. The tampon product according to paragraph A1, wherein the grip region comprises greater than or equal to six circumferentially formed faces.

A9. The tampon product according to paragraph A1, wherein the grip region comprises greater than or equal to eight circumferentially formed faces.

A10. The tampon product according to any one of the preceding paragraphs, wherein each of the faces of the grip region project inwardly toward the longitudinal applicator axis.

A11. The tampon product according to any one of the preceding paragraphs, wherein the grip region comprises greater than four outwardly projecting ribs, wherein each of the ribs are positioned between the faces.

A12. The tampon product according to paragraph A11, wherein each of the faces has a face circumferential width and each of the ribs has a rib circumferential width, wherein the face circumferential width is greater than the rib circumferential width.

A13. The tampon product according to any one of the preceding paragraphs, wherein each of the faces is the same shape.

A14. The tampon product according to any one of the preceding paragraphs, wherein the faces of the grip region are substantially flat.

A15. The tampon product according to any one of the preceding paragraphs, wherein each of the faces of the grip region are at least one of concave and convex.

A16. The tampon product according to any one of the preceding paragraphs, wherein the faces of the grip region comprise one or more textures.

A17. The tampon product according to any one of the preceding paragraphs, wherein the outer member comprises an end region disposed adjacent to the grip region, wherein the end region comprises an internal end region diameter, wherein the internal end region diameter is less than an internal barrel diameter, and wherein an external inner member diameter is at least 90% of the internal end region diameter.

B18. A tampon product comprising: an applicator comprising an outer member and an inner member slidably engaged with the outer member, the outer member comprising an outer surface, a barrel region, and a grip region, wherein the applicator comprises one or more layers, wherein at least one of the one or more layers comprises cellulosic material, and wherein the grip region comprises greater than four faces.

B19. The tampon product according to paragraph B18, wherein the outer member comprises an insertion end region disposed opposite to the grip region.

B20. The tampon product according to any one of the preceding paragraphs, wherein the one or more layers are spirally wound to form at least one of the outer member and the inner member.

B21. The tampon product according to any one of the preceding paragraphs, wherein the grip region comprises greater than four ribs, wherein each of the ribs are positioned between the faces.

B22. The tampon product according to any one of the preceding paragraphs, wherein each of the faces extends from an upper grip region to a lower grip region.

B23. The tampon product according to paragraph B22, wherein each of the faces have a radius of curvature.

B24. The tampon product according to paragraph B23, where each of the radius of curvature of each of the faces are the same.

B25. The tampon product according to paragraph B22, wherein the radius of curvature of at least two of the faces are the same.

B26. The tampon applicator according to paragraph B18, wherein the applicator comprises an end region disposed adjacent to the grip region, wherein the end region comprises a rolled edge.

B27. The tampon product according to any one of the preceding paragraphs, comprising a tampon, wherein the tampon comprises a pledget and a cord attached to the pledget, wherein the pledget is disposed within at least a portion of the barrel region of the applicator.

B28. The tampon product according to paragraph B27, wherein at least a portion of the cord is braided.

B29. The tampon product according to paragraph B27, wherein the inner member comprises a first end region and a second end region opposite the first end region, wherein the first end region of the inner member is configured to engage a portion of the pledget, and wherein the second end region comprises a flange.

B30. The tampon product according to any one of the preceding paragraphs, wherein each of the one or more layers comprise the cellulosic material.

B31. The tampon product according to paragraph B18, wherein at least one of the one or more layers consists of cellulosic material.

B32. The tampon product according to paragraph B27, wherein the pledget has an expulsion force of less than 800 gf.

C33. A tampon product comprising: an applicator comprising an outer member and an inner member slidably engaged with the outer member, the outer member comprising an outer surface, a barrel region, and a grip region, wherein the applicator comprises one or more layers, wherein each of the one or more layers comprises cellulosic material, and wherein the grip region comprises greater than four faces.

C34. The tampon product according to paragraph C33, wherein the cellulosic material is paper.

C35. The tampon product according to paragraph C33, wherein the cellulosic material comprises pulp.

C36. The tampon product according to any one of the preceding paragraphs, wherein the applicator comprises less than 10 percent by weight petroleum, petroleum derivatives and/or plastics with petroleum or petroleum-derived components or precursor materials.

C37. The tampon product according to any one of the preceding paragraphs, wherein each of the greater than four faces extends from an upper grip region to a lower grip region.

C38. The tampon product according to paragraph C37, wherein each of the greater than four faces have a radius of curvature that is about 300 mm.

C39. The tampon product according to paragraph C37, wherein each of the greater than four faces have a radius of curvature that is from about 4.3 mm to about 900 mm.

C40. The tampon product according to any one of the preceding paragraphs, wherein an end region of the outer member and the grip region of the outer member contact the inner member at two contact points that are spaced apart along a longitudinal applicator axis.

C41. The tampon product according to paragraph C33, wherein the applicator exhibits a water content of from 0% to about 20%.

C42. The tampon product according to paragraph C33, wherein the applicator exhibits a water content of from 1% to about 15%.

C43. The tampon product according to paragraph C33, wherein the applicator exhibits a water content of from 4% to about 10%.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon product comprising:
an applicator comprising an outer member and an inner member slidably engaged with the outer member, the outer member comprising an outer surface surrounding a longitudinal applicator axis, a barrel region, and a grip region, wherein the grip region comprises greater than four circumferentially formed faces.

2. The tampon product of claim 1, comprising a tampon, wherein the tampon comprises a pledget and a cord attached to the pledget, wherein the pledget is disposed within at least a portion of the barrel region of the applicator.

3. The tampon product of claim 1, wherein the barrel region has an external barrel diameter and the inner member has an external inner member diameter, and wherein the external inner member diameter is less than 70% of the external barrel diameter.

4. The tampon product of claim 1, wherein the grip region has a first internal grip diameter and a second internal grip diameter, wherein the first internal grip diameter is greater than the second internal grip diameter.

5. The tampon product of claim 4, wherein the inner member has an external inner member diameter, and wherein the external inner member diameter is greater than 90% of the second internal grip diameter of the grip region.

6. The tampon product of claim 4, wherein the difference between the first internal grip diameter and the second internal grip diameter is at least about 2 mm.

7. The tampon product of claim 1, wherein the outer member comprises an insertion end region opposite the grip region.

8. The tampon product of claim 1, wherein the grip region comprises greater than or equal to six circumferentially formed faces.

9. The tampon product of claim 1, wherein the grip region comprises greater than or equal to eight circumferentially formed faces.

10. The tampon product of claim 1, wherein each of the faces of the grip region project inwardly toward the longitudinal applicator axis.

11. The tampon product of claim 1, wherein the grip region comprises greater than four outwardly projecting ribs, wherein each of the ribs are positioned between the faces.

12. The tampon product of claim 11, wherein each of the faces has a face circumferential width and each of the ribs has a rib circumferential width, wherein the face circumferential width is greater than the rib circumferential width.

13. The tampon product of claim 1, wherein each of the faces is the same shape.

14. The tampon product of claim 1, wherein the faces of the grip region are substantially flat.

15. The tampon product of claim 1, wherein each of the faces of the grip region are at least one of concave and convex.

16. The tampon product of claim 1, wherein the outer member comprises an end region disposed adjacent to the grip region, wherein the end region comprises an internal end region diameter, wherein the internal end region diameter is less than an internal barrel diameter, and wherein an external inner member diameter is at least 90% of the internal end region diameter.

17. A tampon product comprising:
an applicator comprising an outer member and an inner member slidably engaged with the outer member, the outer member comprising an outer surface, a barrel region, and a grip region,
wherein the applicator comprises one or more layers, wherein at least one of the one or more layers comprises cellulosic material, and
wherein the grip region comprises greater than four faces.

18. The tampon product of claim 17, wherein the one or more layers are spirally wound to form at least one of the outer member and the inner member.

19. The tampon product of claim 17, wherein the grip region comprises greater than four ribs, wherein each of the ribs are positioned between the faces, and wherein each of the faces extends from an upper grip region to a lower grip region, and wherein each of the faces have a radius of curvature.

20. A tampon product comprising:
an applicator comprising an outer member and an inner member slidably engaged with the outer member, the outer member comprising an outer surface, a barrel region, and a grip region,
wherein the applicator comprises one or more layers, wherein each of the one or more layers comprises cellulosic material, and
wherein the grip region comprises greater than four faces, and wherein each of the greater than four faces have a radius of curvature that is from about 4.3 mm to about 900 mm.
